(19) United States
(12) Patent Application Publication
(10) Pub. No.: US 2023/0225964 A1
(43) Pub. Date: Jul. 20, 2023

(54) MICROSTIRRING DEVICES AND TECHNIQUES FOR ENHANCING BIOAVAILABILITY OF ORALLY ADMINISTERED DRUGS

(71) Applicant: The Regents of the University of California, Oakland, CA (US)

(72) Inventors: Joseph Wang, San Diego, CA (US); Rodolfo Andres Mundaca Uribe, La Jolla, CA (US); Berta Esteban-Fernandez de Avila, San Diego, CA (US); Ronnie Fang, San Diego, CA (US); Liangfang Zhang, San Diego, CA (US)

(21) Appl. No.: 18/156,328

(22) Filed: Jan. 18, 2023

Related U.S. Application Data
(60) Provisional application No. 63/300,594, filed on Jan. 18, 2022.

(51) Int. Cl. A61K 9/00; A61J 3/06; A61K 9/46; A61K 9/16; A61K 9/20

(52) U.S. Cl. CPC A61K 9/0053; A61J 3/06; A61K 9/0007; A61K 9/1611; A61K 9/2018

(57) ABSTRACT

Disclosed are methods, materials and devices that pertain to a microstirring pill technology with built-in mixing capability for oral drug delivery that greatly enhances bioavailability of its therapeutic payload. In some aspects, a drug delivery device includes a pill matrix dissolvable in a fluid medium and loaded with a plurality of drug payloads; and a plurality of micro stirrers embedded in the pill matrix and configured to create a local fluid transport upon interacting with a biological fluid surrounding the microstirring pill.

### PRIORITY CLAIM AND CROSS-REFERENCE TO RELATED APPLICATIONS

This patent document claims the priority and benefits of U.S. Provisional Application No. 63/300,594, titled “MICRO STIRRING DEVICES AND TECHNIQUES FOR ENHANCING BIOAVAILABILITY OF ORALLY ADMINISTERED DRUGS” filed on Jan. 18, 2022. The entire content of the aforementioned patent application is incorporated by reference as part of the disclosure of this patent document.

### STATEMENT REGARDING FEDERALLY SPONSORED RESEARCH OR DEVELOPMENT

This invention was made with government support under grant numbers R21NS114764 and CA200574 awarded by the National Institutes of Health (NIH). The government has certain rights in the invention.

### TECHNICAL FIELD

The disclosed technology generally relates to drug delivery technologies.

### BACKGROUND

Gastric acid, primarily including hydrochloric acid produced by parietal cells in the gastric glands, plays a role in maintaining the stomach's digestive function. It enables gastric proteolysis by denaturing proteins from food for break down by digestive enzymes. It also inhibits the growth of many microorganisms that enter the stomach and thus reduces the risk of pathogen infection. However, the harsh gastric environment may also create a physiological barrier in the stomach for the use and delivery of therapeutic drugs, such as protein-based drugs and some antibiotics. In these cases, the drugs may be combined with a proton pump inhibitor (PPI) to reduce the production of gastric acid. The effectiveness of PPIs may be due to the irreversible binding to the proton pumps to suppress acid secretion for approximately 12 to 24 hours. Long-term use of PPIs can cause adverse side effects such as headache, diarrhea and fatigue, and in more serious scenarios can cause anxiety and depression, as well as server reaction rhabdomyolysis. Due to these problems, alternative approaches are needed that can temporarily neutralize gastric acid without causing adverse side effects.

### SUMMARY

Disclosed are methods, materials and devices that pertain to a microstirring pill technology with built-in mixing capability for oral drug delivery that greatly enhances bioavailability of its therapeutic payload.

In some implementations of the disclosed technology, a drug delivery device includes a pill matrix dissolvable in a fluid medium and loaded with a plurality of drug payloads; and a plurality of micro stirrers embedded in the pill matrix and configured to create a local fluid transport upon interacting with a biological fluid surrounding the microstirring pill.

In some implementations of the disclosed technology, a micromotor-based substance-delivery pill device includes a pill matrix including one or more biocompatible materials dissolvable in a fluid medium; a plurality of payload substances dispersed within the pill matrix; and a plurality of micromotor particles dispersed within the pill matrix and operable to create a stirring effect within the fluid medium upon dissolution of the pill matrix in the fluid medium and release of the plurality of payload substances to accelerate distribution of the plurality of payload substances.

In some implementations of the disclosed technology, a method for administering a drug delivery device includes forming a drug delivery device by combining a plurality of microstirrers and a pill matrix that is dissolvable in a fluid medium and is loaded with a plurality of drug payloads; and administering the drug delivery device to a patient to allow the drug delivery device to release the plurality of microstirrers and the plurality of drug payloads into at least one of a gastric fluid or an intestinal fluid of the patient, wherein the plurality of microstirrers creates a local fluid transport in the at least one of a gastric fluid or an intestinal fluid of the patient to provide an accelerated distribution of plurality of drug payloads.

### BRIEF DESCRIPTION OF THE DRAWINGS

FIGS. 1A-1F show in vitro dissolution rate and self-stirring capability of microstirring pills implemented based on some embodiments of the disclosed technology.

FIGS. 2A-2G show a self-stirring effect of example microstirrers on tracer particles and drug payloads based on some embodiments of the disclosed technology.

FIGS. 3A-3C show in vivo acetylsalicylic acid (ASA) delivery using an example microstirring pill in a murine model based on some embodiments of the disclosed technology.

FIGS. 4A-4D show in vivo ASA delivery using an example microstirring pill in a porcine model based on some embodiments of the disclosed technology.

FIG. 5 shows scanning electron microscopy (SEM) images of an example Mg-based micro stirrer and energy-dispersive X-ray spectroscopy (EDX) images illustrating the distribution of elemental Mg and Ti.

FIG. 6 shows time-lapse images showing the dissolution of a static pill and an example microstirring pill (e.g., 10% microstirrers) in 0.7 M HCl solution stirred at 200 rpm.

FIG. 7 shows diffusion coefficients of tracers only, tracers in a pill, and tracers in a microstirring pill in simulated gastric fluid.

FIG. 8A shows an example experimental design for quantifying acetaminophen (APAP) dissolution. FIG. 8B shows a comparison of dissolution profiles of APAP between static and microstirring pills made with laboratory prepared excipients (top) and commercial excipients (bottom).

FIG. 9 shows time-lapse images showing the dissolution of a static pill and of a microstirring pill.

FIG. 10 shows ASA absorbed fraction for static and microstirring pills.

FIG. 11 shows a schematic of the preparation of ASA-loaded microstirring pills.

FIG. 12 shows an example motor pill with built-in stirring capabilities towards enhanced drug release and distribution in gastrointestinal (GI) tract.

FIGS. 13A and 13B show in vitro motor pill characterization.

FIGS. 14A and 14B show in vivo micromotor pill actuation.

FIG. 15 shows an example of metformin microstirring pill concept towards enhanced drug efficacy towards management of type 2 diabetes.

FIGS. 16A-16C show in vivo studies in a murine animal model of metformin microstirring pills efficacy.

FIG. 17 shows an example method for administering a drug delivery device based on some embodiments of the disclosed technology.

### DETAILED DESCRIPTION

Section headings are used in the present document only to improve readability and do not limit scope of the disclosed embodiments and techniques in each section to only that section.

Disclosed are methods, materials and devices that pertain to a microstirring pill technology with built-in mixing capability for oral drug delivery that greatly enhances bioavailability of its therapeutic payload. In some embodiments, the microstirring technology can be applied to various biomedical applications.

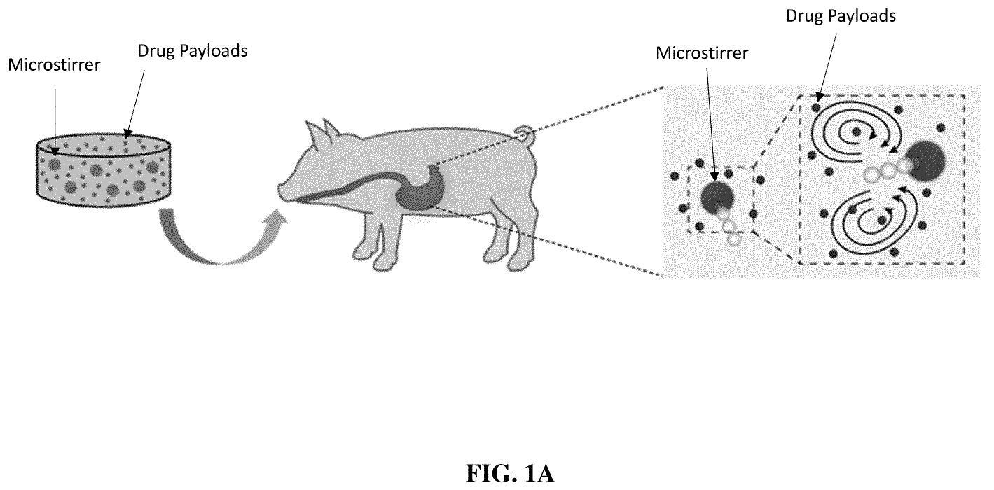

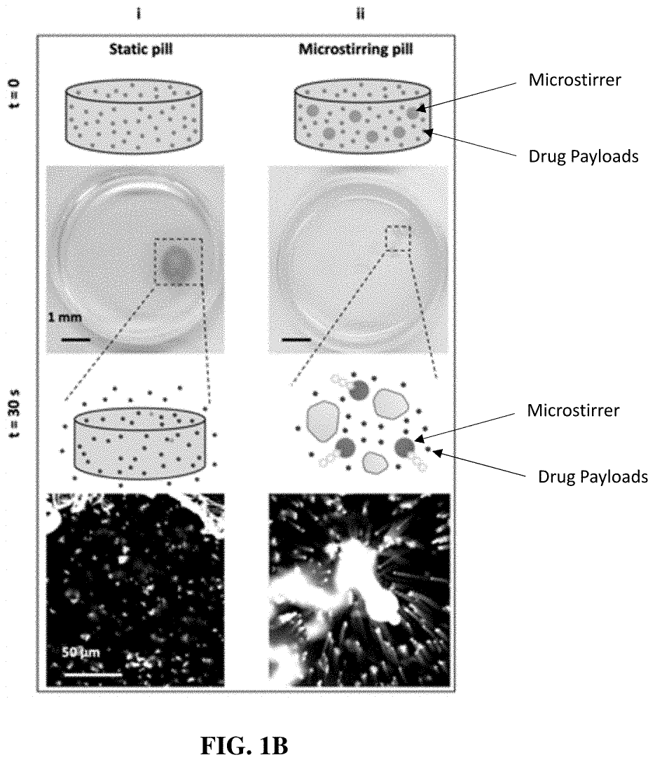

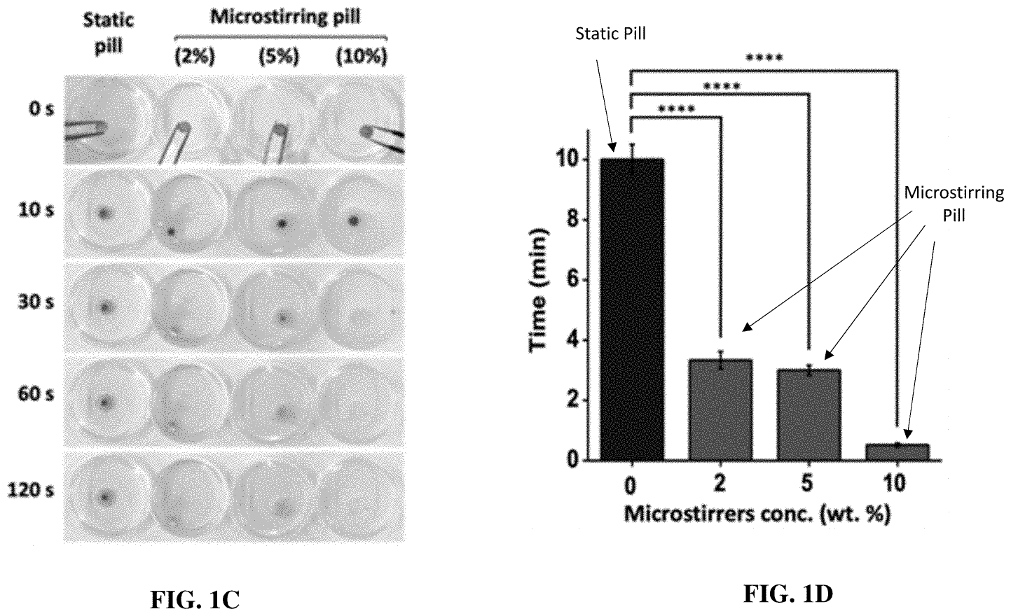

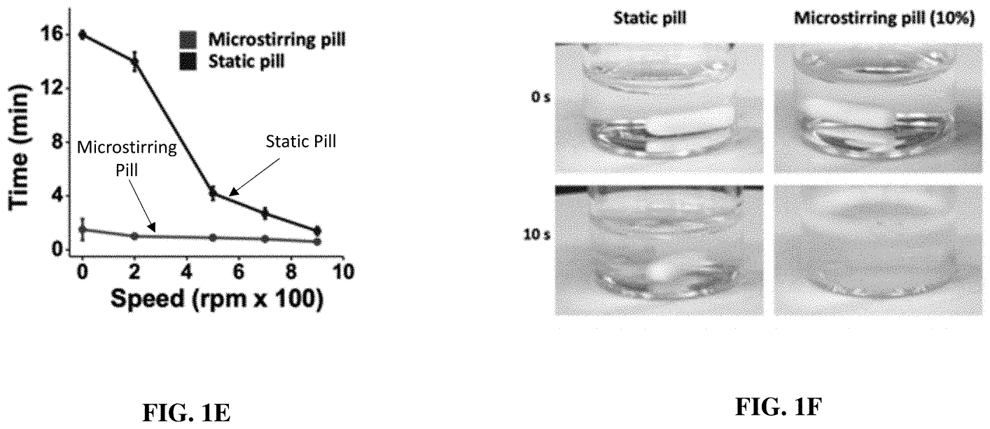

FIGS. 1A-1F show in vitro dissolution rate and self-stirring capability of microstirring pills implemented based on some embodiments of the disclosed technology. FIG. 1A is a schematic illustration of the self-stirring and mixing capability of a microstirring pill after in vivo administration. FIG. 1B shows schematics and images of (i) static and (ii) microstirring pills, demonstrating the faster dissolution rate of microstirring pills and their improved payload dispersion. FIG. 1C shows time-lapse images showing the dissolution of a static pill and microstirring pills (prepared with 2%, 5%, and 10% of microstirrers, by mass) in 2 mL of 0.7 M HCl solution. FIG. 1D shows a comparison of dissolution times of static and microstirring pills (prepared with different microstirrer loadings) in 0.7 M HCl solution. (one-way ANOVA, ****p<0.0001). FIG. 1E shows a comparison of dissolution times of static and microstirring pills (prepared with 10% microstirrers) in 0.7 M HCl solution under stirring conditions (0 to 900 rpm). FIG. 1F shows time-lapse images displaying the dissolution of a static pill and a microstirring pill (10% microstirrers) after 10 s in a stirred 0.7 HCl solution at 200 rpm.

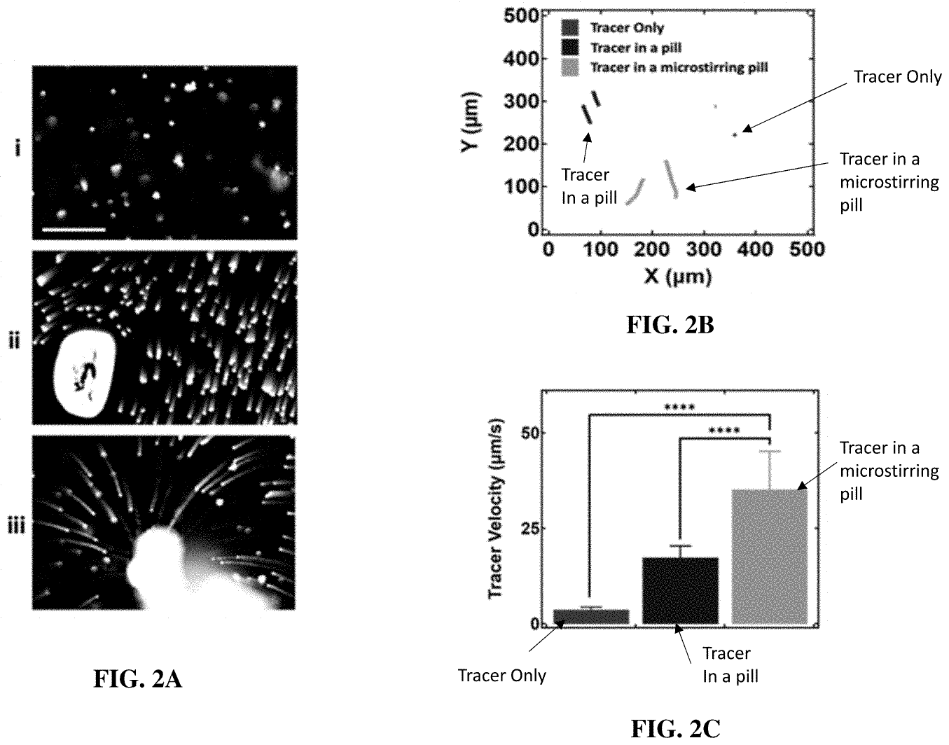

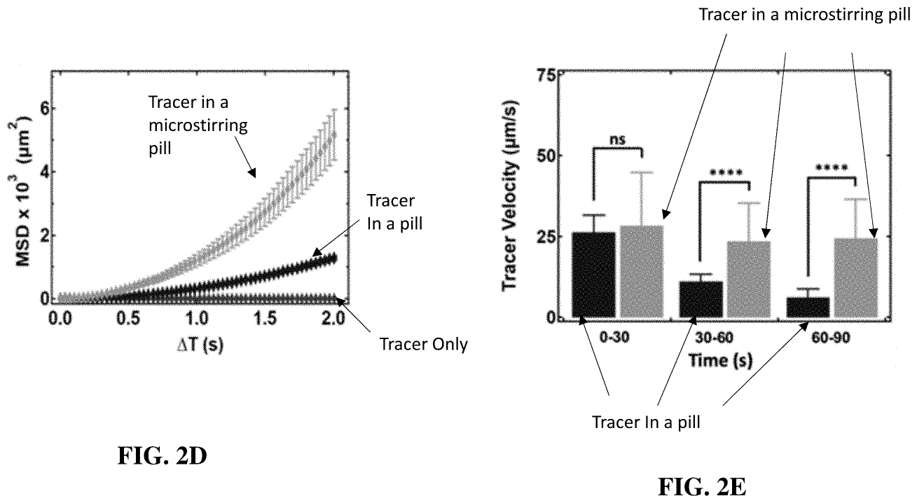

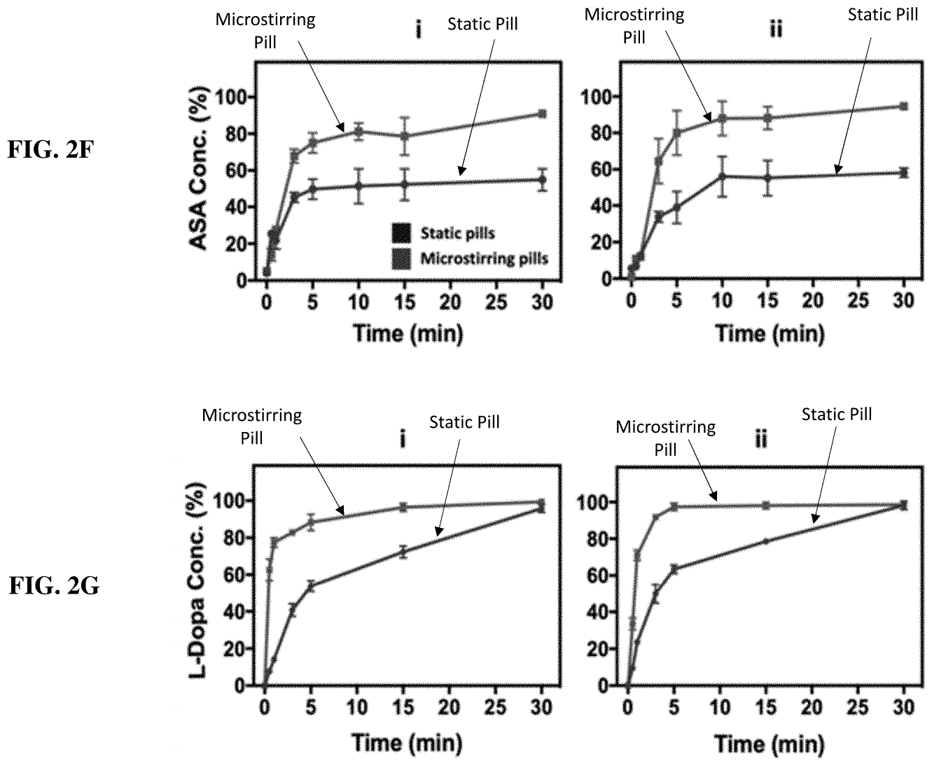

FIGS. 2A-2G show a self-stirring effect of example microstirrers on tracer particles and drug payloads based on some embodiments of the disclosed technology. FIG. 2A shows a visualization of fluid mixing generated by overlapping a stack of 30 color-inverted bright-field images corresponding to 1 s of motion: (i) tracer particles alone in gastric fluid; (ii) tracer particles loaded into a static pill in gastric fluid; and (iii) tracer particles loaded into a microstirring pill in gastric fluid (scale bar: 50 μm). FIG. 2B shows optical trajectories corresponding to FIG. 2A. FIG. 2C shows velocity of tracer particles over a representative 2 s duration from the midpoint of the microstirrer lifetimes (one-way ANOVA, ****p<0.0001). FIG. 2D shows mean squared displacement (MSD) of tracer particles alone in solution, released from a static pill, and released from a microstirring pill from the same representative 2 s duration in FIG. 2C. FIG. 2E shows velocity of tracer particles for representative 30 s durations over the microstirrer lifetimes (one-way ANOVA, ****p<0.0001; ns=not significant). FIG. 2F shows a comparison of in vitro dissolution profiles of aspirin (ASA) between static and microstirring pills made with (i) lab oratory prepared excipients and (ii) commercial excipients. FIG. 2G shows a comparison of in vitro dissolution profiles of levodopa (L-Dopa) between static and microstirring pills made with (i) laboratory prepared excipients and (ii) commercial excipients.

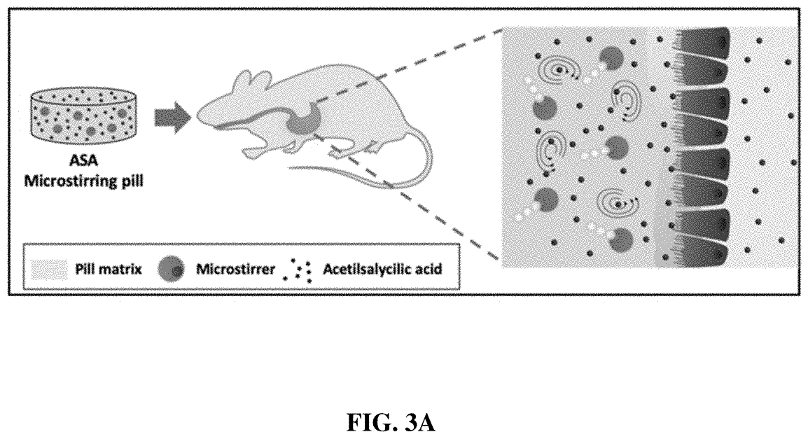

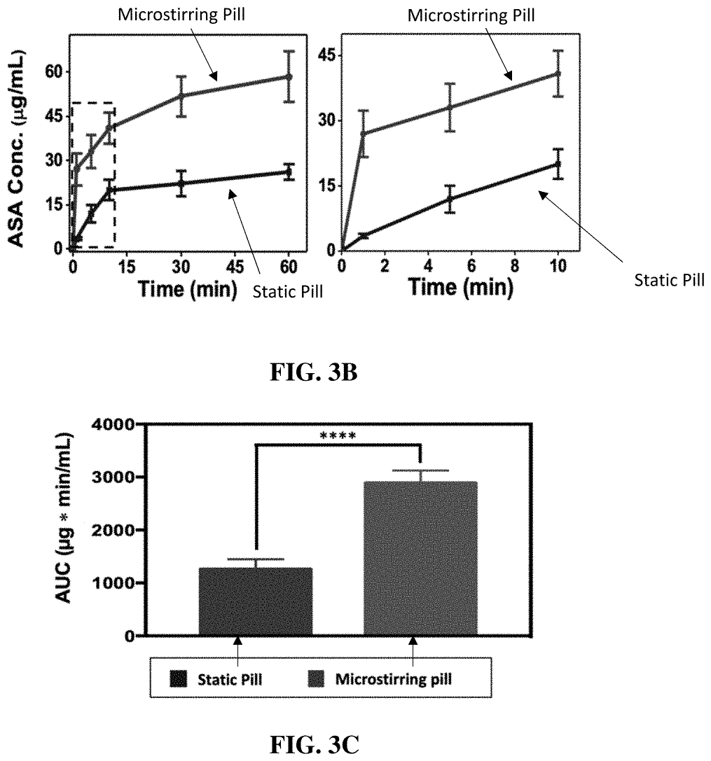

FIGS. 3A-3C show in vivo aspirin (ASA; acetylsalicylic acid) delivery using an example microstirring pill in a murine model based on some embodiments of the disclosed technology. FIG. 3A shows a schematic showing the concept for in vivo ASA absorption kinetic study using static and microstirring pills (ASA, 0.6 mg). FIG. 3B shows serum concentration of ASA after administration of static pills and microstirring pills (n=6) (left: complete kinetic profiles over 60 min; right: kinetic profiles over the initial 10 min). FIG. 3C shows ASA AUC values for static and microstirring pills over 60 min. Unpaired Student's t-test, ****p<0.0001.

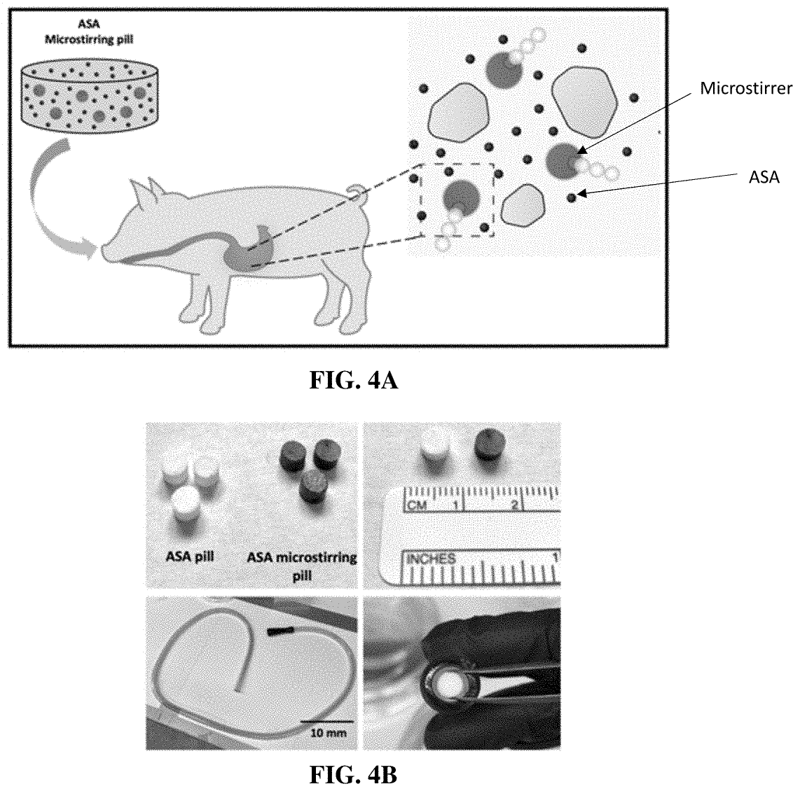

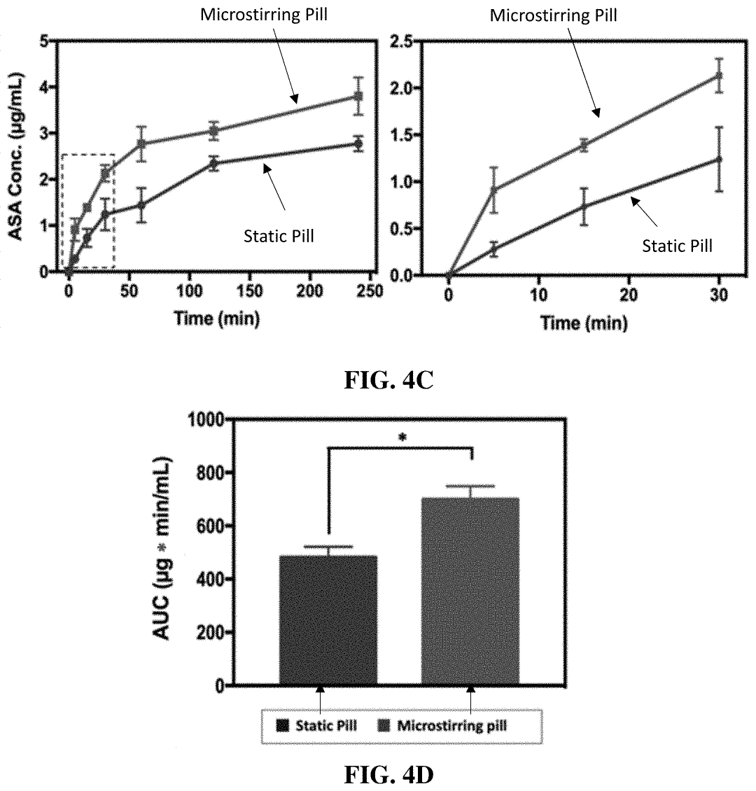

FIGS. 4A-4D show in vivo ASA delivery using an example microstirring pill in a porcine model based on some embodiments of the disclosed technology. FIG. 4A shows a schematic of microstirring pill technology and its application in a porcine model. FIG. 4B shows images of ASA-loaded static and microstirring pills (top row) and the tube used to perform the pill administration by oral gavage (bottom row). FIG. 4C shows serum concentration of ASA after administration of ASA-loaded microstirring pills and static pills (n=3) (left: complete kinetic profile over 250 min; right: kinetic profile over the initial 30 min). FIG. 4D shows ASA AUC values for static and microstirring pills over 4 h (unpaired student's t-test, *p<0.05).

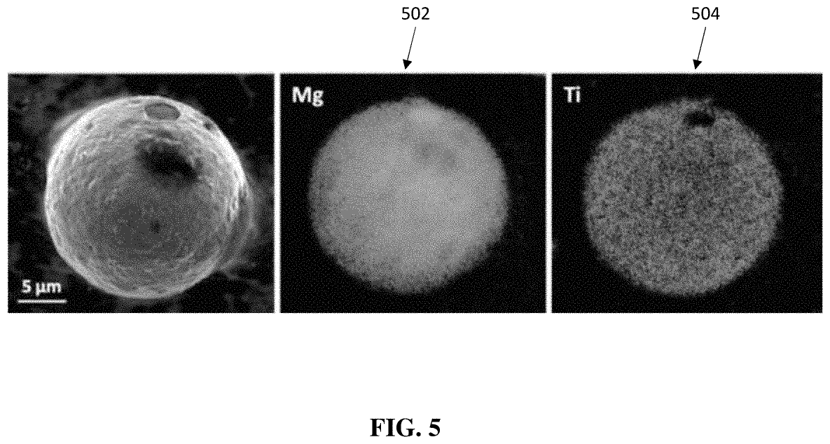

FIG. 5 shows scanning electron microscopy (SEM) images of an example Mg-based microstirrer and energy-dispersive X-ray spectroscopy (EDX) images illustrating the distribution of elemental Mg (502) and Ti (504).

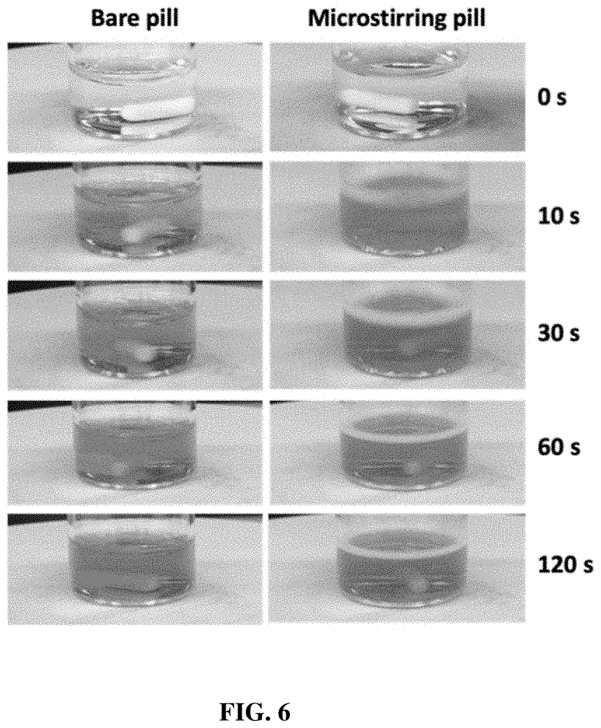

FIG. 6 shows time-lapse images showing the dissolution of a static pill and a microstirring pill (10% microstirrers) in 0.7 M HCl solution stirred at 200 rpm.

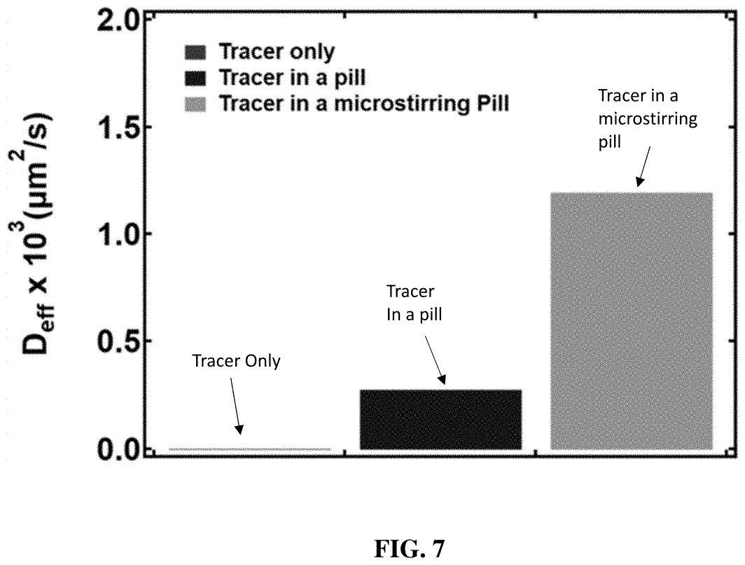

FIG. 7 shows diffusion coefficients of tracers only, tracers in a pill, and tracers in a microstirring pill in simulated gastric fluid.

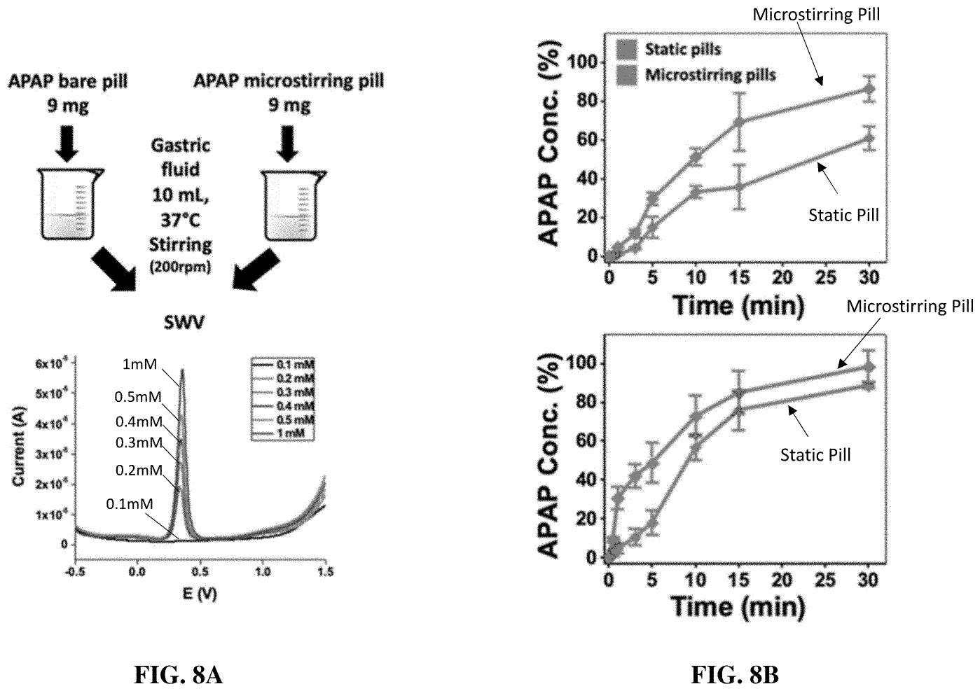

FIG. 8A shows an example experimental design for quantifying acetaminophen (APAP) dissolution. FIG. 8B shows a comparison of dissolution profiles of APAP between static and microstirring pills made with laboratory prepared excipients (top) and commercial excipients (bottom).

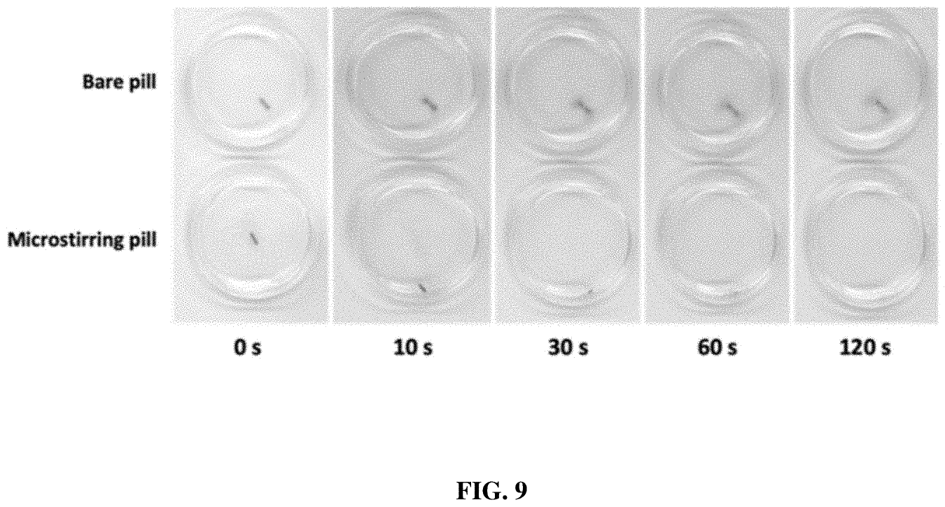

FIG. 9 shows time-lapse images showing the dissolution of a static pill and of a microstirring pill (16% microstirrers) in 2 mL of 0.7 M HCl solution. This formulation is used for the in vivo studies in mice.

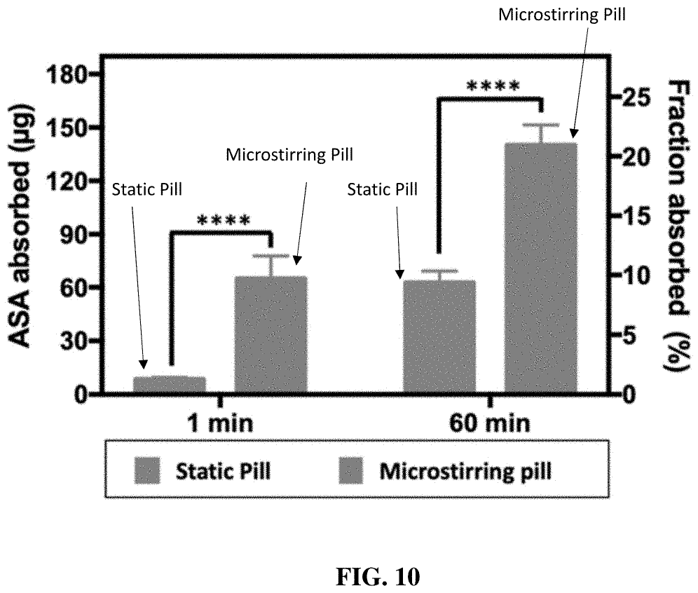

FIG. 10 shows ASA absorbed fraction for static and microstirring pills at 1 and 60 min after administration.

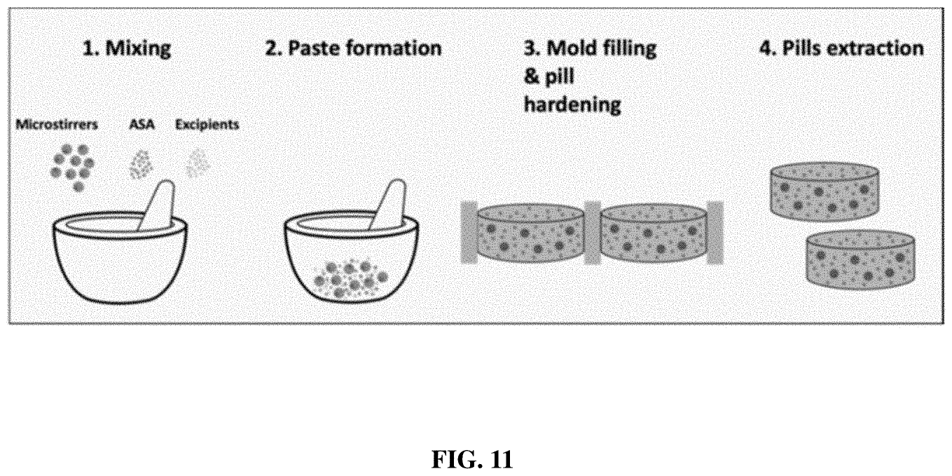

FIG. 11 shows a schematic of the preparation of ASA-loaded microstirring pills.

The disclosed technology can be implemented in some embodiments to provide a novel microstirring pill platform technology which possesses built-in mixing capability for in vivo oral drug delivery, leading to significantly enhanced drug absorption and bioavailability. In some embodiments, the in vitro dissolution profiles at different microstirrer loadings and different fluid stirring speeds demonstrate a substantially faster release of several model drugs at a speed that emulates gastric motility. By loading microstirring pills with tracer particles, the enhanced local fluid transport due to the pumping effect that is exerted. Microstirring pills, loaded with aspirin, are orally administered to both mice and pigs. The acid-driven propulsion and self-stirring effect of the released micromotors in the stomach environment led to greatly enhanced aspirin uptake and bioavailability compared to static aspirin pills. Since the encapsulated drugs and microstirrers are decoupled, the drug loading is not affected or compromised by the microstirrer excipient These findings are of considerable relevance towards drug delivery in humans, particularly when considering the encouraging results obtained using the porcine model. In some embodiments, the microstirring pill platform may be adapted to enhance delivery to other regions of the GI tract, including the small intestines. Furthermore, the microstirrer pill technology implemented based on some embodiments may be applied to the delivery of a wide range of different therapeutic cargoes, such as peptides, proteins, or oral vaccines. Overall, microstirring pills may help to bridge the microengine field with the pharmaceutical industry, and self-stirring excipient may be used in state-of-the-art pill formulations to modulate drug bioavailability upon oral administration.

The disclosed nano-/micro-motor pill platform provides built-in stirring capabilities for enhanced in vivo drug delivery, distribution and absorption, e.g., using self-propelled nanomotors and/or micromotors. The disclosed technology can be implemented in some embodiments to incorporate self-propelled micromotors (e.g., based on biocompatible and biodegradable materials such as magnesium (Mg) or zinc (Zn) bodies) into the pill matrix. For example, once a pill dissolves in gastrointestinal (GI) fluid, the nano- and/or micro-motors are activated, inducing self-mixing of the loaded drugs, thus enhancing retention, distribution, and absorption of loaded drugs in the GI tract.

In some embodiments, aspirin and acetaminophen may be used as example model drugs, yet this motor pill technology may be applied to all types of drugs. The example Mg-based micromotors are used for validation; however, other types of micromotors with different actuation mechanisms (e.g., acoustic, magnetic, electrical), including, but not limited to, Zn-based nano/micromotors, gold-based nano/micromotors, and iron-based nano/micromotors, among others, may be used as excipients for fabricating other types of motor pills. In some implementations, the Mg-based micromotors are chosen as the initial pill excipients because Mg can self-react in gastric and intestinal fluids. Such self-propelled micromotors not only provide self-stirring of the loaded drug, but also enhance distribution and accelerate absorption, thus improving bioavailability of the drug. The therapeutic payload and the motors are decoupled, with the motors solely serve as micro-stirrers in the biological fluids and thus facilitate the mixing distribution, and retention, to improve the drug bioavailability. Such decoupling allows also not to compromise the drug loading (compared to drug loaded motors).

The disclosed technology can be implemented in some embodiments to enhance in vitro release of aspirin and acetaminophen, two common painkillers, which require of fast absorption and for enhanced in vivo distribution and absorption of aspirin using a mouse model. Aspirin is also widely used for response to potential heart attack. Studies using other animal models are currently in progress. This motor pill technology can also be broadly applied for the in vivo GI delivery of a variety of drugs with different characteristics. Another advantage of this approach is that, by using motors as an excipient material in pill format, drug loading does not have to be compromised.

The disclosed technology can be implemented in some embodiments to provide synthetic micromotors with built-in stirring capabilities for enhancing in vitro and in vivo drug distribution and absorption.

Micro/nanomotors, based on different propulsion mechanisms, such as chemical, acoustic, magnetic, and electrical, among others, are capable of moving in different biological fluids while performing a variety of tasks. Specifically, micromotors utilized for in vivo GI delivery are composed of reactive cores (Mg or Zn) and an inert shell or outer surface (e.g., titanium dioxide (TiO₂), gold, or various polymers, among others), along with an opening on one or various sides of the motor to enable exposure of the reactive core to the biological fuel. These micromotors are biocompatible and in some cases completely biodegradable. The autonomous reaction of such micromotors in the GI fluids enables them to rapidly propel in these biofluids and widely distribute. Such active characteristics make them excellent for inducing a stirring motion, which can help to enhance release and distribution of the loaded drugs, so that they can be rapidly absorbed, thus reducing the drug action time.

Cargo-loaded active micromotors can be incorporated into orally-ingestible tablets, based on common pharmaceutical formulations and excipient materials (e.g., lactose and maltose), for localized gastric delivery and easier oral administration. However, it is noted that in previous studies, the built-in stirring capabilities of such micromotors are not explored in that work neither in vitro nor in vivo. Also, in earlier work, the amount of therapeutic cargo per pill is compromised by the loading efficiency of the micromotor.

In some embodiments, the microstirring pill platform takes advantage of the stirring and mixing capabilities of self-propelled micromotors for enhanced release and distribution of the loaded drugs, thus accelerating their absorption in the corresponding location of the GI tract. The motor pill stirring platform is not limited to one type of drug and can be easily extended to multiple types of therapeutics. Other modes of motor actuation are also considered, including magnetic, acoustic, electric and biological, among others.

At least one enhancement of the disclosed microstirrer pill technology platform from other micromotor-based drug delivery systems is that, in other micromotor-based drug delivery systems, the therapeutic payloads are typically loaded onto the motors. For example, previous strategies have shown that, during the propulsion, the motors directly carry their payloads to destination. However, in some implementations of the disclosed technology, the therapeutic agents and the motors are decoupled, where the motors solely serve as micro-stirrers in the biological fluids and thus facilitate the mixing, distribution, and retention. Ultimately, the present platform can improve drug bioavailability.

This disclosure describes the technique to combine micromotor technology and pills to develop a new active drug delivery system with rapid drug distribution and absorption. Different from other drug delivery approaches that passively travel through the GI tract, these motor pills rapidly distribute the drug in the GI tract, enhancing its absorption and reducing the actuation time. Such motor pills with built-in stirring represent an important advantage for the potential treatment of many diseases and conditions that require fast action of the drugs.

In some example embodiments, a micromotor pill system includes a pill matrix (e.g., which can be made of lactose and maltose or other biocompatible substance), which is loaded with a drug (e.g., aspirin and acetaminophen are used in the example studies as model painkillers) and further incorporates Mg- or other micromotors as active stirrers. The example micromotor pill is orally administered, and once in the target destination (e.g., stomach or intestine), the pill starts dissolving and releasing the active micromotors. Such Mg-motors react with the GI fluids, thus inducing efficient self-stirring of the drug and accelerating its distribution and absorption.

In some implementations, the disclosed pill system can accelerate pill dissolution and enhance drug retention and absorption, thus yielding faster increasing and higher blood drug concentrations. In order to gain insights into the effect of self-stirring induced by micromotors on pill dissolution and drug release, an in vitro dissolution profile can be obtained. Two groups of aspirin (ASA) pills can be fabricated, e.g., bare pills (lactose/maltose matrix) as a reference, and micromotor pills (lactose/maltose matrix, Mg-micromotors 10%); then, pills from both groups are immersed into 10 mL of gastric fluid simulant under stirring at 200 rpm at 37° C. Aliquots are taken at 0, 1, 3, 5, 10, 15, and 30 minutes after starting the experiment, and dissolved aspirin is quantified by an ELISA kit. The example Mg-micromotor incorporation allows better pill disintegration and better aspirin dissolution, as is shown at 5 minutes, e.g., where ASA concentration is about 2 times higher for the motor pill formulation. After 30 min, motor pills released about 90% of the initial ASA, while bare pills released less than 60%. After having studied the micromotor pills under in vitro conditions, this platform is tested in vivo. In some embodiments of the disclosed technology, Mg-based micromotors as active stirrers can improve drug absorption and bioavailability.

In some embodiments, as shown in FIG. 3A, a motor pill, loaded with a drug, is orally administered, and then, the pill is disintegrated and micromotors are released and activated, starting the self-stirring process, which allows better mixing of the drug and enhanced absorption. In an example in vivo implementation, two groups of CD1 mice are administered with either ASA 0.6 mg bare pills or ASA 0.6 mg motor pills. Blood samples are taken after 0, 1, 5, 10, 30 and 60 minutes after administration by oral gavage. In some implementations, as shown in FIG. 3B, the kinetic profile for ASA bare pills and ASA motor pills shows that ASA blood concentration is about 9 times higher for the motor pill group at 1 minute after the oral administration. After 60 minutes, ASA blood concentration is still about 1.4 times higher with the motor pill group. These example results clearly show that this invention is able to substantially enhance the absorption of ASA, especially during the first 10 minutes after administration. Furthermore, bioavailability is enhanced, which is reflected in both higher absorption speed and maximum drug blood concentration (Cmax).

The disclosed technology can be implemented in some embodiments to provide microstirring pills that can enhance bioavailability of orally administered drugs.

Majority of drugs are administered orally, yet their efficient absorption is often difficult to achieve, with a low dose fraction reaching the blood compartment. Here, a microstirring pill technology based on some embodiments can provide built-in mixing capability for oral drug delivery that greatly enhances bioavailability of its therapeutic payload. Embedding microscopic stirrers into a pill matrix enables faster disintegration and dissolution, leading to improved release profiles of three widely used model drugs, aspirin, levodopa, and acetaminophen, without compromising their loading. Unlike recently developed drug-carrying nanomotors, drug molecules are not associated with the microstirrers, and hence there is no limitation on the loading capacity. These embedded micro stirrers are fabricated through the asymmetric coating of titanium dioxide thin film onto magnesium microparticles. In vitro tests illustrate that the embedded microstirrers lead to substantial enhancement of local fluid transport. In vivo studies using murine and porcine models demonstrate that the localized stirring capability of microstirrers leads to enhanced bioavailability of drug payloads. Such improvements are of considerable importance in clinical scenarios where fast absorption and high bioavailability of therapeutics are critical. The encouraging results obtained in porcine model suggest that the microstirring pill technology has translational potential and can be developed toward practical biomedical applications.

Oral drug formulations are widely used due to ease of administration, high patient compliance and safety, and cost-effective manufacturing. Nevertheless, the oral delivery route has some inherent disadvantages when compared with other methods of administration, including reduced control over the drug release rate, limited target specificity and absorption across the mucosal barrier, drug degradation in the gastrointestinal (GI) tract, and side effects due to the high dose required for achieving the desired therapeutic effect. One of the main parameters to assess drug performance is bioavailability, which is defined as the fraction of dosed drug that reaches systemic circulation. Achieving high bioavailability depends strongly on the drug solubility, GI absorption, and permeability, and often involves high dosing and undesired side effects. Thus, extensive efforts have been dedicated to enhancing drug bioavailability not only by modifying drug molecules themselves but also by developing formulation systems. For instance, nanotechnology has been utilized to increase bioavailability of certain drugs. One approach encapsulated insulin inside polymeric nanoparticles for sustained delivery through oral administration, with the size and large surface area of nanoparticles leading to improved absorption and the therapeutic index.

Pills are solid drug formulations, comprised of functional active pharmaceutical ingredients and inactive excipients in powder, crystalline, or granular forms, that are commonly manufactured by compression techniques. Once a pill is ingested, it disintegrates in the stomach upon contacting GI fluids and releases its drug payload, followed by drug absorption in either the stomach or intestine. The pill disintegration, drug dissolution, and dispersion processes, and GI permeability, have profound impacts on the degree of drug absorption and on the bioavailability of the therapeutic payload. Several pharmaceutic strategies have been developed in pills to improve the drug bioavailability, such as a pullulan-based pill loaded with rosuvastatin flexible chitosomes, a cefdinir-cyclodextrin complex in tablets for improved drug dissolution rate, and orodispersible tablets. In addition, effervescent pills, which generally contain a mixture of acid salts and carbonate ion salts that release carbon dioxide upon contact with water, can facilitate faster absorption. Moreover, “smart pills/capsules” have been introduced recently, including a luminal unfolding microneedle injector pill for insulin delivery, a capsule for oral once-weekly drug delivery system for human immunodeficiency virus (HIV) treatment, and a capsule for monitoring GI health.

The disclosed technology can be implemented in some embodiments to provide a unique microstirring pill platform technology with built-in in situ stirring capability for oral drug delivery with enhanced drug uptake and bioavailability. In some embodiments, the inclusion of chemically-powered microstirrers into a pharmaceutical pill will enhance the drug dissolution and dispersion in the stomach fluid, leading to faster absorption and increased bioavailability. In some embodiments, magnesium (Mg)-based Janus microparticles (often called “microengines”) with self-propulsion ability can be used to fabricate microstirrers. Such microstirrers consist of 25-μm Mg microparticles, partially coated with a thin titanium dioxide (TiO₂) layer, where the fabrication process leaves a small opening for microbubbles to exit and propel the microparticle upon reacting with appropriate chemical fuel. Furthermore, Mg is an excellent candidate for use in the body as there is high tolerance for ionic Mg in vivo and excess Mg can be easily removed or absorbed. Major developments in the field of synthetic microengines over the past decade have led to important advances that can benefit medicine. Synthetic microengines, capable of converting energy into mechanical motion, can be powered by different sources including chemical, magnetic, electric, and optical, among others. Chemically powered microengines have gained particular attention for in vivo biomedical applications, mainly due to their autonomous self-propulsion, and they have shown benefits for enhanced delivery of therapeutic cargoes with deeper tissue retention.

These Mg microstirrers are incorporated as an excipient into a solid lactose/maltose pill. Note that in the microstirrer pill formulation the therapeutic drugs and the microstirrers are decoupled, which allows for high drug loading while providing efficient stirring action that enhances the drug release, distribution, and absorption once the pill reaches the stomach. A series of in vitro characterizations demonstrate the effective mixing capability of the Mg microstirrers under static and dynamic conditions, along with their ability to enhance local fluid transport. In vivo studies using a murine animal model demonstrate that the in situ stirring capability of Mg microstirrers offers enhanced absorption and bioavailability, and this leads to a faster elevation of serum drug levels. Experimental demonstration and verification of the microstirrer pill in a porcine model, which is physiologically closer to humans, further highlight the translational potential of this technology.

Overall, these findings show that by co-encapsulating Mg microstirrers as an excipient to a pill formulation along with therapeutic drugs, the microstirrer pill can effectively modulate and enhance the bioavailability of common orally delivered drugs both immediately post administration and at longer time scales. Unlike previous studies where synthetic microengines are loaded with therapeutic agents to perform active delivery, in this platform Mg microstirrers are not associated with the payloads, allowing for a better drug distribution and absorption; moreover, there is no more limitation in the payload loading than the pill capacity, and the fabrication process is simpler compared to older systems. As the use of microstirrers is independent of the loaded drugs, such microstirrer pill can be a platform technology broadly applicable for numerous types of oral drugs.

Microstirring Pill Concept and In Vitro Self-Stirring Capabilities

As illustrated in FIG. 1A, the overall microstirrer pill concept is that Mg-based microstirrers (e.g., FIG. 5) react in acidic gastric conditions to generate gas microbubbles, thus inducing a stirring effect which leads to significantly faster pill dissolution and rapid dispersion of the drug payload. In addition to the microstirrers and drugs, the pill consists of a matrix formed from a biocompatible combination of lactose and maltose. To evaluate and demonstrate the capabilities of the microstirring pill strategy, we selected three model drugs, aspirin (ASA;

acetylsalicylic acid), levodopa (L-Dopa), and acetaminophen (APAP). FIG. 1B illustrates the overall ability of the Mg microstirrers to induce self-stirring in solution that leads to a faster pill dissolution rate when compared to static pills (traditional pills without microstirrers in their composition). The schematic illustrations and corresponding images show the dissolution process of a static pill and a microstirring pill in 0.7 M hydrochloric acid (HCl) solution. The optical images taken after 30 s of immersion in the HCl solution clearly demonstrated the rapid microstirring pill dissolution, as indicated from the uniform diffusion of the yellow dye. On the contrary, static pills dissolved significantly slower, with the dye spreading dominated by passive diffusion. The microscopy images show the pill formulations with tracer particles, which are used to visualize the fluid mixing effect exerted by the encapsulated Mg microstirrers.

To gain insights into the effect of such self-stirring on pill disintegration and dissolution, dye-loaded pills with different microstirrer loadings (2, 5, and 10 wt %) are prepared and further tested in 0.7 M HCl (FIG. 1C). Time-lapse photographic imaging is used to visualize the dissolution process and corresponding dye diffusion at different times ranging from 0 to 120 s. As displayed by the images, just 10 s post-immersion in HCl solution, the yellow dye coming from microstirring pills permeated a significant portion of the petri dish volume, whereas the dye coming from the static pill is restricted to the pill perimeter. Another difference observed when using microstirring pills is the presence of gas microbubbles, reflecting the efficient reaction of the Mg microstirrers within the acidic solution. As expected, the pill dissolution process is dependent on the loading of the microstirrers and time, as indicated from the even distribution of the yellow dye in the images taken at 120 s. Pills containing 2%, 5%, and 10% microstirrers are dissolved 3.0, 3.3, and ten times faster than static pills, respectively (FIG. 1D).

Aiming at mimicking the dissolution of the microstirring pill under the natural movement of the gastric environment, the microstirring pill dissolution is further evaluated under dynamic conditions. In some embodiments, the dissolution time of static pills and microstirring pills (prepared with 10% microstirrers) are compared at different external fluid stirring speeds ranging from 0 to 900 rpm (FIG. 1E). The microstirring pill formulation exhibited a dissolution profile that is significantly faster than that of the static pill in general. As expected, the gap between the two pills decreased at higher fluid stirring speed values. Notably, the 10.7-fold faster pill dissolution time when working at 200 rpm, which is a speed that simulates the fluid hydrodynamics exerted on hydrophilic tablets within the GI, suggested that the microstirring pills could induce faster pill dissolution in an in vivo setting. Time-lapse imaging further illustrated the different dissolution rates of static and microstirring pills after 10 s in 0.7 M HCl and at 200 rpm (FIG. 1F; FIG. 6). While the microstirring pill is almost completely dissolved, the static pill maintained most of its structure after the same period. Overall, these in vitro findings demonstrate that microstirring pills offer faster dissolution profiles with enhanced payload dispersion when compared to the corresponding static pills.

Self-Stirring Effect of Microstirrers on Tracer Particles and Drug Payloads

To further elucidate the role of the microstirrers in shortening the pill dissolution times, tracer particles are employed to extract important mixing parameters. Polystyrene tracer particles, 2 μm in size, are loaded into static and microstirring pills, and their positions are tracked overtime. To illustrate the differences in tracer particle motion, 30 sequential images are stitched together from a video capture of a dissolution event (FIG. 2A). Tracer particles alone in dissolution media only experienced Brownian motion and thus did not exhibit “tails.” For the static pill, short tails are visualized, which resulted from the convective flows associated with pill dissolution. The tails displayed by tracers released from the microstirring pill are significantly longer due to the increased convective flow associated with the microstirrers. This is also reflected when tracking individual tracers over a representative 2 s duration taken from the midpoint of a microstirrer's lifetime, where it could be seen that those released from the microstirring pill had substantially larger displacement (FIG. 2B). In terms of average velocity, there is also a ten times difference between tracers released from a microstirring pill versus controls placed directly into dissolution media. To further describe these differences, we calculated the mean square displacement (MSD) versus delay time (ΔT) for each of the three scenarios from the representative 2 s durations in FIG. 2C. The linear nature of the control tracers in simulated gastric fluid confirmed their Brownian motion behavior. The MSD of the tracers in a static pill and microstirring pill exhibited a parabolic trend with MSD≈ΔT² (FIG. 2D). This superdiffusive behavior suggested departure from Brownian motion and enhanced transport at the microscale. To assess the self-stirring capability of the microstirrers, we extracted effective diffusion coefficients for the tracer particles, which are calculated based on Equation (1).

MSD(ΔT)=2×d×D_{eff}×ΔT (1)

where d is the dimensionality of the system (in this case d=2) and D_{eff} is the effective diffusion coefficient. Using the maximum slope of the curves, we estimated that tracers experiencing purely Brownian motion had a D_{eff} value of 0.74 μm² s⁻¹. Tracers released from the static pill had a larger D_{eff} value of 279 μm² s⁻¹ while tracers being actively stirred by microstirrers exhibited a D_{eff} value of 1197 μm² s⁻¹. Such significant differences in D_{eff} clearly illustrate the enhanced motion of the tracers associated with the convective flows of pill dissolution, with the effect being more pronounced in the case of the microstirring pill (FIG. 7). To gain further insights into the overall performance of the microstirrers over their whole lifetime, we analyzed tracer motion at different time points during the pill dissolution (FIG. 2E). We observed that the speed of the tracers released from a static pill decreased with time, confirming that the convective flows moving the tracers are mainly due to the dissolution of the pill. On the other hand, the velocity of the tracers released from the microstirring pill remained relatively constant and is consistently higher than those from a static pill in all three 30 s intervals during the lifetime of the microstirrers. This observation suggested that microstirrers contribute to enhanced fluid mixing not only on short time scales (representative 2 s durations from the midpoint of their lifetime, FIG. 2C) but also over the entire pill dissolution timeframe (representative 30 s durations over the microstirrer lifetime, FIG. 2E).

Subsequently, the microstirrer self-stirring effect is evaluated by assessing the dissolution profiles of various drugs. In this case, we studied ASA, L-Dopa, and APAP, three widely used medicines for antiplatelet therapy, Parkinson's disease treatment, and as an analgesic/antipyretic, respectively, where fast drug absorption is essential. In some embodiments, microstirrers are incorporated along with ASA, L-Dopa, or APAP into pills, and the dissolution profiles are compared to the ones obtained from static pills loaded with the same drugs. For our laboratory preparation, a pill matrix consisting of only maltose and lactose is used. In order to evaluate the self-stirring effect in commercial-type formulations, separate pills are also prepared by triturating ASA, Sinemet, and Tylenol pills, incorporating microstirrers into the mixture, and then compressing all excipients to fabricate new pills. The same procedure is followed for static commercial pills without adding microstirrers. Each of the prepared pills is dissolved in 10 mL of gastric fluid simulant at 37° C. Then, aliquots are taken at different times during an interval of 30 min to quantify the concentration of ASA by ELISA, or L-Dopa and APAP by square wave voltammetry (FIGS. 2F, 2G, and 8A, 8B). A faster dissolution can be achieved when microstirrers are in the pill formulations, demonstrating higher drug release at each time point for all scenarios. For ASA, the profiles for microstirring laboratory and commercial-type pills are similar in shape, and about 90% of the drug is dissolved after 30 min, which is about 1.6-fold higher than that for the static pills. A slightly different behavior is observed for the pills loaded with L-Dopa. Whereas full drug dissolution is not observed for static pills loaded with ASA, full release is achieved for static L-Dopa pills, albeit the kinetics are significantly delayed compared to their microstirrer counterparts. Such behavior likely reflects the higher solubility of L-Dopa in water compared to ASA (66 and 3 mg mL⁻¹, respectively). The same trends are obtained when APAP microstirring pills are tested. Overall, the inclusion of microstirrers to the pill formulations enabled a faster release of the drug, which could be crucial in certain emergency medical applications.

In Vivo Evaluation of Microstirring Pills in a Murine Model

With the microstirring pills providing faster release of ASA, L-Dopa, and APAP, we performed a suite of in vivo studies using murine and porcine models. The goal of these animal studies is to evaluate whether the self-stirring effect could help to accelerate the absorption of orally delivered drugs, and, as a consequence, offer higher bioavailability and faster pharmacokinetic uptake profiles. ASA is chosen as the model drug for these in vivo studies because it is absorbed both from the stomach and from the upper intestinal tract. For the murine model based on some embodiments, 1×3 mm microstirring pills are prepared, and their disintegration and dissolution capabilities are tested, as shown in FIG. 9. FIG. 3A illustrates the concept of the in vivo study performed using the murine model. Mice (n=6) are administered with static or microstirring pills, both containing 0.6 mg ASA. Then, blood samples are collected at 1, 5, 10, 30, and 60 min post-administration in order to quantify the ASA concentrations (FIG. 3B). From the first time point, a significant difference between the serum drug concentrations is observed, with the value being ≈8.0-fold higher when microstirring pills are administered. The improved drug bioavailability persisted over the entire monitoring period, with the final serum ASA levels plateauing at greater than double the concentration achieved with static pills. The amount of absorbed ASA (FIG. 10) is calculated on the basis of a mouse weight of ≈20 g and the corresponding circulating blood volume of about 2.4 mL. The absorbed fraction obtained with microstirring pills 60 min post-administration is about 2.2-fold higher than the one obtained with static pills (23.4% and 10.4% of the administered dose, respectively), reflecting a greater fraction of the administered dose that reached the circulation using the microstirring pills. A significantly larger difference in absorbed fraction (about 8-fold) is observed 1 min after the drug administration (FIG. 10), with 10.8% and 1.4% of administrating the microstirring and static pills, respectively. Such behavior is particularly important in emergency medical situations, when high blood drug concentrations are needed immediately after the pill ingestion. Specifically, due to its antiplatelet properties, ASA is highly recommended for immediate treatment of patients suspected of having a heart attack. FIG. 3C shows the area under the curve (AUC) values obtained with static and microstirring pills at 60 min post-administration. The AUC obtained with ASA microstirring pills is about 2.4-fold higher than with ASA pills (2890 versus 1260 μg min mL⁻¹, respectively), reflecting a larger dose fraction of the drug reaching the systemic circulation over this period of time using microstirring pills. These findings here clearly demonstrate that the use of microstirrers as an excipient in a pill formulation greatly enhances drug absorption and bioavailability, resulting in both accelerated and elevated serum concentrations.

In Vivo Evaluation of Microstirring Pills in a Porcine Model

Based on the encouraging results in the murine model, the ASA absorption in vivo study is extended to a porcine model. As a larger animal, pigs display more similarity to humans with regards to the GI tract and are commonly used for predicting human bioavailability of orally administered drugs. The general concept of this experiment is illustrated in FIG. 4A, in which a microstirring ASA pill is administered to a pig; when the pill reaches the stomach, it starts to dissolve while the microstirrers are activated, allowing for a faster dissolution and a greater drug dispersion due to the in situ self-stirring effect. Pills are fabricated by forming in a mortar a paste composed of ASA, microstirrers, and the pharmaceutical excipients lactose and maltose, followed by a hardening process within an appropriately sized mold (FIG. 11). The resulting 3×5 mm pills are designed to fit in the oral gavage feeding tube that is used for the pill administration (FIG. 4B). When looking at the kinetic profiles obtained from this study (FIG. 4C), a similar trend is observed compared with those of the mouse study. The ASA blood concentration at the first 5 min time point is 3.26 times higher when microstirrers are included inside the pill, and it remained and 1.72-fold higher at 15 and 30 min post-administration, respectively. After 4 h of the administration, the ASA concentration obtained with microstirring pills is ≈1.4 times higher. FIG. 4D shows the AUC values obtained with static and microstirring pills during this study. The AUC obtained with ASA microstirring pills is higher than with ASA pills (698.9 versus 481.5 μg min mL⁻¹), indicating that a higher fraction of the drug is absorbed and reached blood circulation over 4 h with microstirring pills. Similar to the murine model, the bloodstream ASA values do not reach the same value at the end of the study as the active pills offer enhanced bioavailability and absorption. These encouraging results, obtained in a porcine model, suggest that the approach of using microstirring pills to enhance drug bioavailability may hold promise toward obtaining similar improvements in humans.

The disclosed technology can be implemented in some embodiments to provide a novel microstirring pill platform technology that possesses built-in mixing capability for in vivo oral drug delivery, leading to significantly enhanced drug absorption and bioavailability. In some embodiments, the in vitro dissolution profiles at different microstirrer loadings and different fluid stirring speeds demonstrate a substantially faster release of several model drugs at a speed that emulates gastric motility. By loading microstirring pills with tracer particles, we demonstrated the enhanced local fluid transport due to the pumping effect that is exerted. Microstirring pills, loaded with ASA, are orally administered to both mice and pigs. The acid-driven propulsion and self-stirring effect of the released microstirrers in the stomach environment led to greatly enhanced ASA uptake and bioavailability compared to static ASA pills. Since the encapsulated drugs and microstirrers are decoupled, the drug loading is not affected or compromised by the microstirrer excipient. These findings are of considerable relevance toward drug delivery in humans, particularly when considering the encouraging results obtained using the porcine model. In the future, the platform could be further adapted to enhance delivery to other regions of the GI tract, including the small intestines. Furthermore, the microstirrer pill technology could be applied to the delivery of a wide range of different therapeutic cargoes, such as peptides, proteins, or oral vaccines. On the other hand, other materials, such as Zn, Fe, Al (and its alloys), and CaCO₃, could be utilized as microstirrers although special considerations must be applied as some of these materials have limited reactivity in intestinal biofluids and lower tolerance in the body compared to Mg.

Overall, microstirring pills may help to bridge the microengine field with the pharmaceutical industry, and self-stirring excipient could one day be used in state-of-the-art pill formulations to modulate drug bioavailability upon oral administration. This simple yet elegant technology has a few unique strengths. First, it has a much higher translation potential: adding only an excipient material (synthetic microstirrers) into the pill formulation without changing anything else; second, it is a platform technology: agnostic to delivered drugs or pill formulations; last, it has tested validated in large animal model, representing the first time for microstirrers (or microengines) to be tested in a large animal model.

Microstirrer Fabrication

Mg-based microstirrers are prepared using commercial Mg microparticles with an average size of 20±5 μm as the core. In order to remove impurities, the Mg microparticles are washed twice with acetone and dried under N₂ current. Then, about 10 mg of Mg microparticles are dispersed onto glass slides previously covered with 100 μL of 0.5% polyvinylpyrrolidone ethanolic solution. The Mg microparticles are then coated with TiO₂ by atomic layer deposition (ALD) at 100° C. for 3000 cycles using an atomic layer deposition system. In this step, the exposed surface of the Mg particle is coated, leaving a small opening at the region where the Mg particles contacted the glass slide. Finally, the Mg microstirrers are retrieved by scratching them off the glass slide.

Microstirrer Characterization

Scanning electron microscopy (SEM) imaging of a Mg microstirrer is obtained with an environmental scanning-electron microscope (ESEM) instrument, using an acceleration voltage of 10 kV. Energy dispersive X-ray analysis (EDX) mapping analysis is performed using an EDX detector attached to the SEM instrument and operated by software.

Pill Preparation

Microstirring pills are prepared by triturating and mixing lactose and maltose in a 60%/40% ratio. Once this mixture is homogeneous, microstirrers (0, 2, 5, or 10% of the total mixture weight) are incorporated and mixed in a mortar, and no changes in the microstirrer structure are observed during this mixing process. Model drugs are added at this step. Subsequently, an ethanol/water wetting solution (75%/25%) is added to the powder mixture to provide a paste-like consistency. In some in vitro experiments, a yellow food dye is added at this stage to facilitate the visualization of the in vitro pill dissolution. Then, the paste is transferred to a cavity plate and each of the cavities is completely filled with the mixture by applying sufficient pressure to ensure tight packing. Immediately after filling the cavities, the cavity plate is lowered onto the peg plate until the wet pills are ejected. Finally, the microstirring pills are allowed to dry and harden over the peg plate at 65° C. for 2 h. Static pills are prepared following the same protocol with the exception of the addition of microstirrers.

Tracer Tracking and Analysis

Tracer tracking experiments are performed by adding 2 μm polystyrene (PS) tracer particles to a solution of gastric acid stimulant with 0.6% Triton X-100 surfactant for the tracer only case (tracer particles are diluted ten times from a stock concentration of 2.62%). For the tracer trajectory analysis for the pill formulations, the same tracer particles are embedded into pills during the fabrication process at a 1.0% loading. Later, pills are dissolved in the same gastric acid simulant containing surfactant. Videos are recorded at 30 fps on an inverted optical microscope coupled with a digital camera. Tracer tracking is performed with software. MSD calculation is performed with the publicly available MATLAB function (ms-danalyzer) for a group of 40 particles (n=40). Overlapped stacks of images are prepared. The stacks correspond to 1 s of motion. The color of the images is inverted to show a black background. Lighter colored tails represent the most recent position of the tracer particle while darker colored tails represent the oldest position.

In Vitro ASA, L-Dopa, and APAP Dissolution Analysis

Pills loaded with ASA, L-Dopa, or APAP are dissolved in 10 mL of gastric fluid simulant under stirring (200 rpm) at 37° C. Aliquots of 25 μL are taken 0, 0.5, 1, 3, 5, 10, 15, and 30 min after the start of the experiment and analyzed for the corresponding drug, as well as after total pill dissolution. ASA is quantified using a salicylates enzyme-linked immunosorbent assay (ELISA) kit following the manufacturer's specifications. L-Dopa and APAP are quantified by using square-wave voltammetry, measuring the anodic peak current corresponding to the oxidation of these drugs. These measurements involved a glassy carbon working electrode, Ag/AgCl reference electrode, and a Pt wire counter electrode, along with a potentiostat.

In Vivo ASA Delivery Study in Mice

To perform the in vivo ASA-loaded microstirring pill delivery study in a murine model, male CD-1 mice are fasted overnight prior to the experiment. Then, mice (n=6) are intragastrically administered with either ASA-loaded microstirring pills or static pills using a stainless-steel X-M dosing syringe. A 50 μL blood sample is collected from the submandibular vein before administration and at 1, 5, 10, 30, and 60 min post-administration. After spinning the blood for 5 min at 3000×g, the serum is collected for quantifying the ASA concentration by ELISA.

In Vivo ASA Delivery Study in Pigs

To perform the in vivo ASA-loaded microstirring pill delivery study in a porcine model, 3 months old female farm pigs 35 kg in weight are fasted overnight prior to the experiment. Then, pigs (n=3) are anesthetized with ketamine, xylazine, and atropine, while monitoring their vital signs. Consequently, pigs are intragastrically administered with either ASA-loaded microstirring pills or static pills using a flexible oral gavage tube. Blood samples are collected from the ear artery before administration and at 5, 15, 30, 60, 120, and 240 min post-administration. After collecting the blood, it is left to clot at room temperature by leaving it undisturbed in a covered tube for 15-30 min; then, samples are centrifuged at 2000×g for 5 min and the serum is collected for ASA quantification by ELISA.

Statistical Analysis

Data are presented as mean±SD. In vitro studies: Statistical analysis of comparison of dissolution times of static and microstirring pills is performed using one-way ANOVA (GraphPad Prism), ****p<0.0001. Error bars represent standard deviation calculated from the dissolution of 3 different pills. Statistical analysis of velocity of tracer particles study is performed using one-way ANOVA, ****p<0.0001 (n=40). In vivo studies in murine model: in the serum concentration of ASA after administration of static pills and microstirring pills study, error bars represent standard deviation calculated from the drug concentration in 6 different mice. In ASA AUC study for static and microstirring pills over 60 min, statistical analysis is performed using unpaired Student's t-test, ****p<0.0001, with n=6. In vivo studies in porcine model: in the serum concentration of ASA after administration of ASA-loaded microstirring pills and static pills statistical study, error bars represent standard deviation calculated from the drug concentration in 3 different pigs. In ASA AUC values for static and microstirring pills over 4 h study, statistical analysis is performed using unpaired Student's t-test, *p<0.05, with n=3. No statistical methods are used to predetermine sample size. Studies are carried out in a non-blinded fashion.

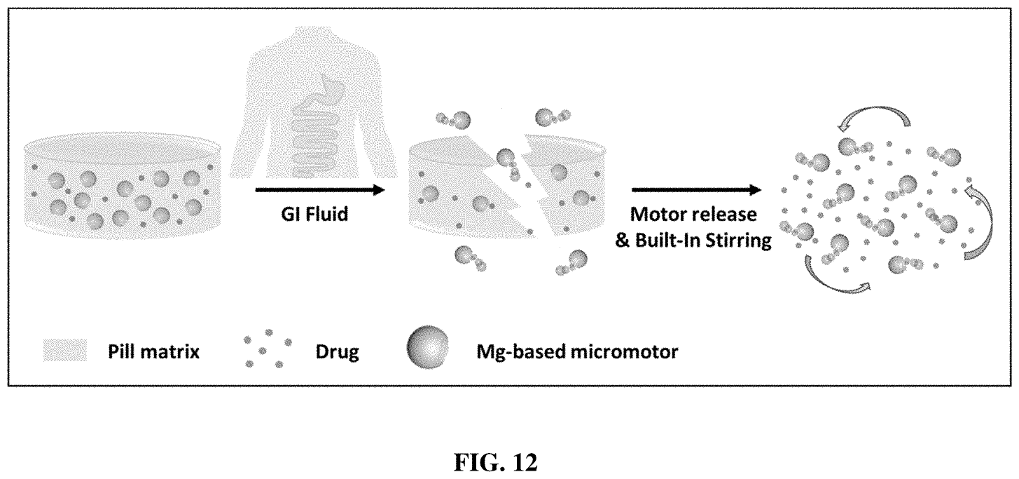

FIG. 12 shows an example motor pill with built-in stirring capabilities towards enhanced drug release and distribution in the GI tract. The micromotors are incorporated within a pill along with common (e.g., lactose/maltose) excipients.

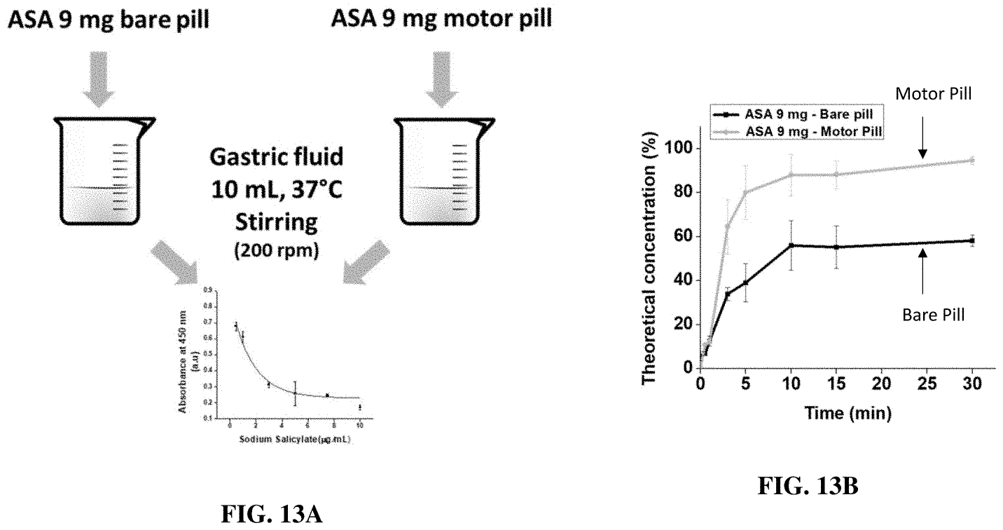

FIGS. 13A and 13B show in vitro motor pill characterization. FIG. 13A shows schematic of experimental design of ASA pill dissolution profile. FIG. 13B shows dissolution profile of ASA bare pills and ASA motor pills (n=3).

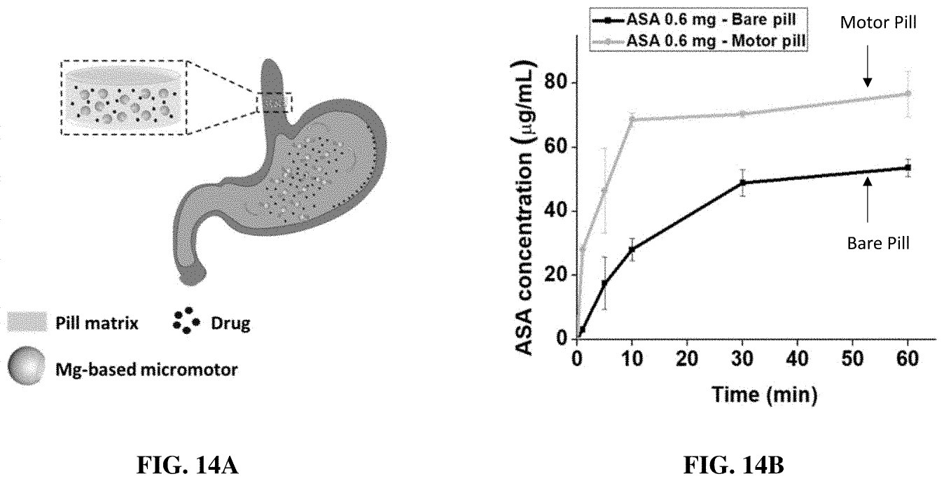

FIGS. 14A and 14B show in vivo micromotor pill actuation. FIG. 14A is schematic that shows the concept of the invention. FIG. 14B show kinetic profile of ASA bare pills and ASA micromotor pills (n=3).

The disclosed technology can be implemented in some embodiments to provide a microstirring pill that enhances the therapeutic efficacy of metformin towards type 2 diabetes treatment.

Here, we incorporated magnesium Mg-based microstirrers to a metformin pill matrix to enhance the therapeutic efficacy of metformin. Such microstirrer pill possess a built-in mixing capability by creating local fluid transport upon interacting with biological fluid to facilitate faster pill disintegration and drug release along with accelerated metformin delivery. In vivo glucose tolerance test utilizing a mouse animal model verified that the metformin microstirring pill significantly enhances the drug therapeutic outcome, displaying the lowest blood glucose levels after a meal. Furthermore, the microstirrers allow for lowering the metformin dosage, still achieving the same therapeutic outcome as the metformin bare pills loaded with the standard dosage. These encouraging results show not only the versatility of this simple yet elegant platform, as they help to improve the payload absorption both in gastric and intestinal environments, but also suggest that this technology aids to enhance the therapeutic outcome of drugs.

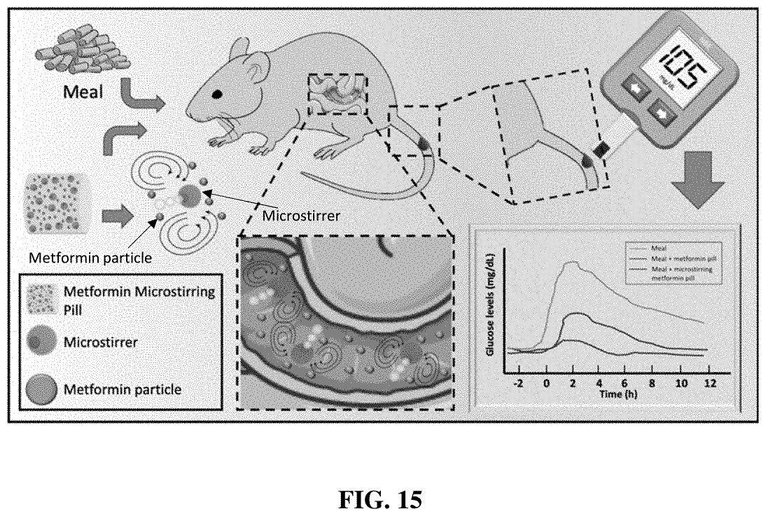

FIG. 15 shows an example of metformin microstirring pill concept towards enhanced drug efficacy towards management of type 2 diabetes.

Referring to FIG. 15, an example metformin microstirring pill, where a mouse, after a meal, is administered with the pill. Upon reaching the small intestine, the microstirrers are activated and lead to faster pill dissolution and drug release, followed by localized convective flow that result with enhanced metformin transport through the intestinal compartment, allowing for an enhanced absorption of the drug. Then, glucose levels are quantified with a glucometer at different timepoints after the metformin microstirring pill administration, resulting in lower glucose spike concentrations when compared with a bare metformin pill control group.

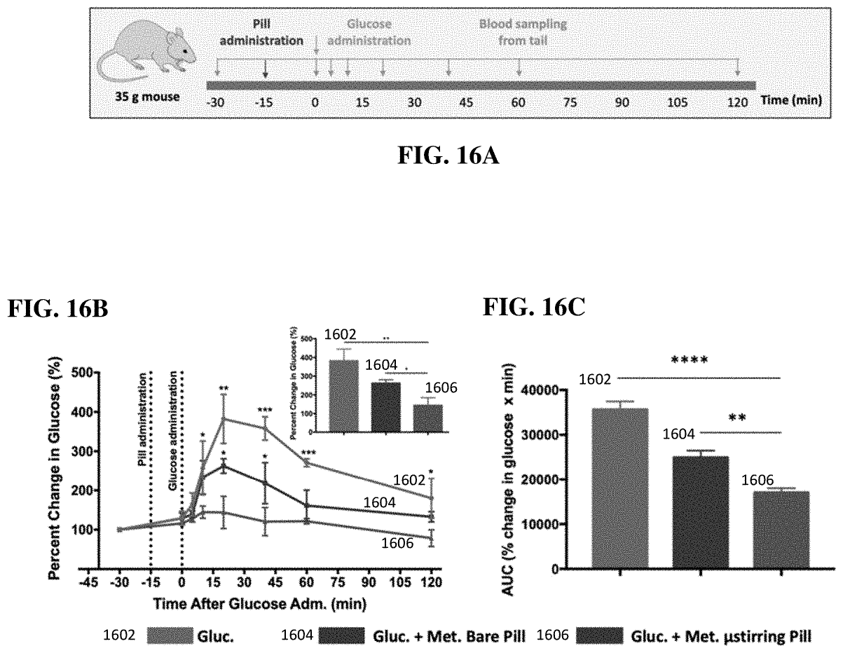

FIGS. 16A-16C show in vivo studies in a murine animal model of metformin microstirring pills efficacy. FIG. 16A shows schematic showing the experimental model, displaying the timeline for pill and glucose administration, and blood sampling. FIG. 16B shows glucose profile, in terms of percent change in glucose, performed in 35 g mice (n=3), comparing the glucose profiles of glucose only, glucose plus metformin bare pill, and glucose plus metformin microstirring pill control groups; inset: comparison of the control groups at the peak glucose level of the oral glucose tolerance test (n=3). Glucose intake: 1.8 g/kg; metformin dose: 120 mg/kg Statistical significance is calculated by one-way ANOVA with Turkey test. FIG. 16C shows glucose AUC values for glucose only, glucose plus metformin bare pills, and glucose plus metformin microstirring pills, over 2 h (n=3). Glucose intake: 1.8 g/kg; metformin dose: 120 mg/kg. Statistical significance is calculated by one-way ANOVA with Turkey test.

FIG. 16A shows an experimental design based on some embodiments of the disclosed technology. Accordingly, about 35 g mice (n=3) are administered with bare or microstirring pills containing 4.2 mg of metformin. The prepared pills are 1×3 mm in dimension, made with lactose and maltose as excipients. Fifteen min after pill administration, a glucose solution is administered to each mouse and the first blood samples are collected from the tail (t=0). The remaining blood samples are collected at 5, 10, 20, 40, 60, and 120 min post glucose administration. Following each collection of blood sample, the glucose levels are quantified using a blood glucose monitor device and the respective glucose profiles are plotted. Furthermore, a control group without any treatment is included in to have a baseline of the animal glucose levels after glucose administration. FIG. 16B shows the glucose profiles for the “Glucose,” “Glucose +Metformin Bare Pill,” and “Glucose+Metformin Microstirring Pill” groups. At 10 min, lower glucose levels are for the microstirring pill group, being reduced in about 50% when compared with the glucose control group, while the bare pill group did in just about 11%. In all the other evaluated timepoints, microstirring pill treatment led to lower glucose levels than the controls, demonstrating better therapeutic efficacy especially between the 20 and 40 min marks, where a significant statistical difference is obtained between the microstirring pill treatment and both controls. As displayed in the inset of FIG. 16B, the peak glucose level obtained 20 min after glucose administration demonstrated a substantial enhancement in metformin efficacy, resulting in about 48% lower glucose levels with the microstirring pills treatment, and about 66% lower than the group with no metformin treatment. This enhanced therapeutic outcome is very important considering the relevant implications this has particularly on T2DM patients where glucose management remains the ultimate goal, mainly due to the strong correlation between glycemic peaks and atherosclerosis, endothelial dysfunction, and cardiovascular disorders. Besides the comparison timepoint by timepoint of all the groups, the area under the curve (AUC), which represents bioavailability of glucose, is calculated and compared, as illustrated in FIG. 16C. The bioavailability of glucose is about 30% lower when microstirring pills are utilized, in compare with metformin loaded in bare pills, and about 50% lower than when no treatment is administered. These results clearly illustrate the perks of our microstirring platform, allowing for an enhanced absorption of the drug, and thus, an improved therapeutic outcome.

In some embodiments, as a result of the enhanced drug absorption and bioavailability, Mg-based micro stirrers help to enhance the therapeutic efficacy of the drug. In addition, combining Mg-based microstirrers with Mg-based micromotors (as drug carriers) allow to realize combinatorial delivery with different kinetic profiles for each of the drugs, as the microstirrers will promote a fast release, distribution, and absorption of the drug.

In some embodiments, a micromotor-based substance-delivery pill device includes a pill matrix comprising one or more biocompatible materials; a plurality of a payload substances dispersed within the pill matrix; and a plurality of micromotor particles dispersed with in the pill matrix, wherein the plurality of micromotor particles are operable as actively-moveable stirrers within a fluid medium when the pill matrix is dissolved to release the dispersed plurality of payload substances and the dispersed plurality of micromotor particles, such that the dispersed plurality of payload substances provide efficient self-stirring of the plurality of payload substances in the fluid medium, thereby accelerating distribution and absorption of the plurality of the payload substance. In one example, the micromotor particles include a core particle and an external coating.

In some embodiments, the fluid medium is a gastrointestinal fluid, and where the plurality of micromotor particles is operable to react with a gastrointestinal fluid. In one example, the micromotor particles include a core particle and an external coating.

In some embodiments, the core particle includes magnesium (Mg), and the external coating includes titanium oxide (TiO2).

In some embodiments, the micromotor particle includes a zinc-based micromotor, gold-based micromotor, or an iron-based micromotor.

In some embodiments, the core particle includes a curved geometry having a diameter in a range of 10 to 30 μm.

In some embodiments, the micromotor particles include a micromotor body including a one or more material layers to provide a structure that surrounds a hollow interior region and has an opening to an exterior of the micromotor body, one or more particles comprising a biocompatible metal element, the one or more particles contained in the interior region of the micromotor body, and a coating coupled to the structure of the micromotor body.

In some embodiments, the pill matrix comprises at least one of lactose, maltose, or other biocompatible substance.

In some embodiments, a method for enhancing substance delivery from a pill device includes a providing a dissolvable pill device comprising a pill matrix formed of one or more biocompatible materials, a plurality of a payload substance, and a plurality of micromotor particles dispersed within the pill matrix, upon ingestion in a stomach, dissolving the pill matrix to release the plurality of the payload substance and the plurality of micromotor particles within a gastric fluid, and stirring, based on at least partially self-propulsion of the micromotor particles, the plurality of payload substances to provide an accelerated distribution and/or absorption of the plurality of the payload substance in the stomach.

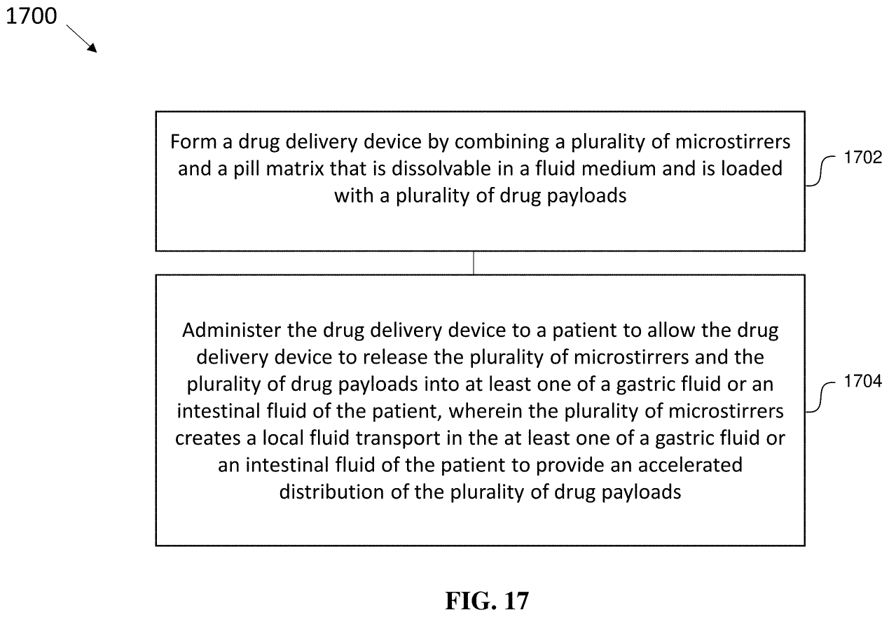

FIG. 17 shows an example method 1700 for operating a drug delivery device based on some embodiments of the disclosed technology.

In some implementations, the method 1700 includes, at 1702, forming a drug delivery device by combining a plurality of microstirrers and a pill matrix that is dissolvable in a fluid medium and is loaded with a plurality of drug payloads, and at 1704, administering the drug delivery device to a patient to allow the drug delivery device to release the plurality of micro stirrers and the plurality of drug payloads into at least one of a gastric fluid or an intestinal fluid of the patient, wherein the plurality of microstirrers creates a local fluid transport in the at least one of a gastric fluid or an intestinal fluid of the patient to provide an accelerated distribution of the plurality of drug payloads.

Therefore, various implementations of features of the disclosed technology can be made based on the above disclosure, including the examples listed below.

Example 1. A drug delivery device comprising: a pill matrix dissolvable in a fluid medium and loaded with a plurality of drug payloads; and a plurality of microstirrers embedded in the pill matrix and configured to create a local fluid transport upon interacting with a biological fluid surrounding the microstirring pill.

Example 2. The device of example 1, wherein the plurality of microstirrers is decoupled from the plurality of drug payloads.

Example 3. The device of example 1, wherein at least one of the plurality of microstirrers includes at least one of magnesium (Mg) or zinc (Zn).

Example 4. The device of example 3, wherein the at least one of the plurality of microstirrers is at least partially coated with a thin titanium dioxide (TiO2) layer.

Example 5. The device of example 1, further comprising a plurality of micromotors operable to carry the plurality of drug payloads.

Example 6. The device of example 5, wherein at least one of the plurality of micromotors includes magnesium (Mg).

Example 7. The device of example 1, wherein the biological fluid includes at least one of a gastric fluid or an intestinal fluid.

Example 8. The device of example 1, wherein the plurality of microstirrers is configured to react in acidic gastric conditions to generate gas microbubbles.

Example 9. The device of example 1, wherein the pill matrix includes at least one of lactose or maltose.

Example 10. A micromotor-based substance-delivery pill device, comprising: a pill matrix including one or more biocompatible materials dissolvable in a fluid medium; a plurality of payload substances dispersed within the pill matrix; and a plurality of micromotor particles dispersed within the pill matrix and operable to create a stirring effect within the fluid medium upon dissolution of the pill matrix in the fluid medium and to release of the plurality of payload substances to accelerate distribution of the plurality of payload substances.

Example 11. The device of example 10, wherein the fluid medium is a gastrointestinal fluid, and where the plurality of micromotor particles is operable to react with the gastrointestinal fluid.

Example 12. The device of example 10, wherein at least one of the plurality of micromotor particles includes a core particle coated with a coating layer.

Example 13. The device of example 12, wherein the core particle includes magnesium (Mg), and the coating layer includes titanium oxide (TiO2).

Example 14. The device of example 10, wherein at least one of the plurality of micromotor particles includes a zinc-based micromotor, a gold-based micromotor, or an iron-based micromotor.

Example 15. The device of example 10, wherein at least one of the plurality of micromotor particles includes a reactive core and a shell with an opening on one or more sides of the shell to enable exposure of the reactive core.

Example 16. The device of example 15, wherein the reactive core includes at least one of magnesium (Mg) or zinc (Zn), and the shell includes at least one of titanium dioxide (TiO2), gold, or a polymer.

Example 17. The device of example 15, wherein the one or more biocompatible materials include at least one of lactose or maltose.

Example 18. A method for operating a drug delivery device, comprising: forming a drug delivery device by combining a plurality of microstirrers and a pill matrix that is dissolvable in a fluid medium and is loaded with a plurality of drug payloads; and administering the drug delivery device to a patient to allow the drug delivery device to release the plurality of microstirrers and the plurality of drug payloads into at least one of a gastric fluid or an intestinal fluid of the patient, wherein the plurality of microstirrers creates a local fluid transport in the at least one of a gastric fluid or an intestinal fluid of the patient to provide an accelerated distribution of the plurality of drug payloads.

Example 19. The method of example 18, wherein the plurality of microstirrers is decoupled from the plurality of drug payloads.

Example 20. The method of example 18, wherein the plurality of microstirrers includes at least one of magnesium (Mg) or zinc (Zn).

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

What is claimed is:

1. A drug delivery device comprising:
a pill matrix dissolvable in a fluid medium and loaded with a plurality of drug payloads; and
a plurality of microstirrers embedded in the pill matrix and configured to create a local fluid transport upon interacting with a biological fluid surrounding the drug delivery device.

2. The device of claim 1, wherein the plurality of microstirrers is decoupled from the plurality of drug payloads.

3. The device of claim 1, wherein at least one of the plurality of microstirrers includes at least one of magnesium (Mg) or zinc (Zn).

4. The device of claim 3, wherein the at least one of the plurality of microstirrers is at least partially coated with a thin titanium dioxide (TiO₂) layer.

5. The device of claim 1, further comprising a plurality of micromotors operable to carry the plurality of drug payloads.

6. The device of claim 5, wherein at least one of the plurality of micromotors includes magnesium (Mg).

7. The device of claim 1, wherein the biological fluid includes at least one of a gastric fluid or an intestinal fluid.

8. The device of claim 1, wherein the plurality of microstirrers is configured to react in acidic gastric conditions to generate gas microbubbles.

9. The device of claim 1, wherein the pill matrix includes at least one of lactose or maltose.

10. A micromotor-based substance-delivery pill device, comprising:
a pill matrix including one or more biocompatible materials dissolvable in a fluid medium;
a plurality of payload substances dispersed within the pill matrix; and
a plurality of micromotor particles dispersed within the pill matrix and operable to create a stirring effect within the fluid medium upon dissolution of the pill matrix in the fluid medium and to release of the plurality of payload substances to accelerate distribution of the plurality of payload substances.

11. The device of claim 10, wherein the fluid medium is a gastrointestinal fluid, and where the plurality of micromotor particles is operable to react with the gastrointestinal fluid.

12. The device of claim 10, wherein at least one of the plurality of micromotor particles includes a core particle coated with a coating layer.

13. The device of claim 12, wherein the core particle includes magnesium (Mg), and the coating layer includes titanium oxide (TiO2).

14. The device of claim 10, wherein at least one of the plurality of micromotor particles includes a zinc-based micromotor, a gold-based micromotor, or an iron-based micromotor.

15. The device of claim 10, wherein at least one of the plurality of micromotor particles includes a reactive core and a shell with an opening on one or more sides of the shell to enable exposure of the reactive core.

16. The device of claim 15, wherein the reactive core includes at least one of magnesium (Mg) or zinc (Zn), and the shell includes at least one of titanium dioxide (TiO₂), gold, or a polymer.

17. The device of claim 15, wherein the one or more biocompatible materials include at least one of lactose or maltose.

18. A method for operating a drug delivery device, comprising:
forming a drug delivery device by combining a plurality of microstirrers and a pill matrix that is dissolvable in a fluid medium and is loaded with a plurality of drug payloads; and
administering the drug delivery device to a patient to allow the drug delivery device to release the plurality of microstirrers and the plurality of drug payloads into at least one of a gastric fluid or an intestinal fluid of the patient, wherein the plurality of microstirrers creates a local fluid transport in the at least one of a gastric fluid or an intestinal fluid of the patient to provide an accelerated distribution of the plurality of drug payloads.

19. The method of claim 18, wherein the plurality of microstirrers is decoupled from the plurality of drug payloads.

20. The method of claim 18, wherein the plurality of microstirrers includes at least one of magnesium (Mg) or zinc (Zn).
